(19) United States
(12) Patent Application Publication
(10) Pub. No.: US 2007/0225697 A1
(43) Pub. Date: Sep. 27, 2007

(54) Apparatus and methods for cardiac ablation

(76) Inventors: Ketan Shroff, Pleasanton, CA (US); Sing Fatt Chin, Pleasanton, CA (US); Amit Agarwal, Mountain View, CA (US); Patrick Morin, Fremont, CA (US)

Correspondence Address: LAW OFFICE OF ALAN W. CANNON, 942 MESA OAK COURT, SUNNYVALE, CA, 94086, US

(21) Appl. No.: 11/388,108

(22) Filed: Mar. 23, 2006

(51) Int. Cl. A61B 18/18; A61B 18/04

(52) U.S. Cl. 606/33

(57) ABSTRACT

An adjustable surgical clamp including one or more ablation elements creates a circular lesion in a first operating mode and a linear lesion in a second operating mode. A “box” lesion surrounding all four pulmonary veins may be formed by using the clamp to create two complimentary C-shaped lesions about pairs of the veins. A thermochromic liquid crystal strip that changes color at a tissue-ablating threshold temperature may be mounted on the surgical clamp to monitor temperature of the ablated tissue. Two microwave antennae may be positioned on the jaws of the clamp relative to each other to produce a combined substantially uniform field of tissue-ablating energy between the jaws.

### FIELD OF THE INVENTION

This invention relates to apparatus and methods for performing cardiac ablation to treat atrial fibrillation, and more particularly to adaptable clamps for forming encircling and linear lesions, approaches to creating uniform tissue-ablating energy fields, and systems for assessing lesion formation.

### BACKGROUND OF THE INVENTION

The ablation of cardiac tissue surrounding the pulmonary veins is a generally accepted surgical method for treatment of atrial fibrillation, particularly in cases where atrial fibrillation has been non-responsive to non-surgical treatment methods or such non-surgical treatment methods have been less than acceptably effective. Ablation of the tissue causes the formation of non-conductive scar tissue that electrically isolates the pulmonary veins. The process of ablating and scarring thus impedes chaotic electrical impulses, originating within the pulmonary veins, from triggering irregular muscular contraction (e.g., fibrillation or flutter) in the cardiac tissue, thereby allowing the heart (e.g., atrium) to contract and pump normally.

Ablation clamps have recently been introduced for use in performing cardiac ablation, for example, as described in U.S. Pat. Nos. 6,546,935 and 6,517,536, and in U.S. Patent Application Publication No. 2004/0106937, each of which are hereby incorporated herein, in their entireties, by reference thereto. The tissue receives ablative energy along the length of the clamp jaws resulting in a continuous lesion created with less effort and time than by using a catheter in a conventional cut and burn approach. Another advantage associated with using a clamp is that squeezing of the tissue between the clamp jaws caused more effective isolation of the ablating element from the blood, thereby reducing the risk of thrombus formation or blood clotting from the ablation. Also, the clamp generally only needs to be positioned once (as opposed to multiple placements and ablations using other techniques) which further reduces the risk of ablating the pulmonary vein itself. Ablation of the pulmonary vein can lead to stenosis. FIG. 1 is a posterior view of a bilateral lesion pattern on a human heart 10 (illustrated without the pericardium, for clarity) used to treat atrial fibrillation and featuring encircling lesions 4,8 made with a clamp and surrounding left 5 and right 7 pulmonary vein ostia, respectively.

Despite these advantages, clamp-created encircling lesions are generally not considered to be sufficient by themselves to ensure electrical isolation, and linear lesions are typically performed to complete the encircling lesions. As shown in FIG. 1, the encircling lesion 4 around the ostia of the left pulmonary veins 5 is connected to the encircling lesion 8 around the right pulmonary veins 7 by a connecting linear lesion 3. Further, linear lesions around the perimeter of the atria 6 and along the length of the aorta 9 may be considered necessary in order to complete the procedure. Additional lesions may also be needed to fill in any non-uniform or discontinuous portions of the encircling lesions created by the ablation clamp. Such lesions cannot be accomplished by existing clamps and a separate ablation tool capable of making the additional lesions 3, 6, 9 (shown in FIG. 1) is commonly required. This requirement necessitates more space in the immediate operating area and complicates the surgical procedure, as different ablation instruments must be alternatively introduced into surgical sites about the heart.

It would be desirable to form both “clamp” (encircling) and linear lesions conveniently. It would also be desirable to ensure that the ablating energy applied by a clamp or similar device from both sides of tissue to be ablated is substantially uniform in order to create a continuous and even lesion and to monitor lesion formation during the ablation process.

### SUMMARY OF THE INVENTION

In accordance with one embodiment of the present invention, a surgical clamp is used to form a cardiac lesion. The clamp comprises a first jaw including a tissue-ablating element disposed to selectively ablate tissue in proximity thereto, and a second jaw detachably coupled to the first jaw that can be adjusted in distance from the first jaw.

In another embodiment of the invention, a single surgical clamp is used to create linear and encircling lesions at a surgical site. The clamp including a pair of jaws is advanced through an incision toward a first portion of the surgical site. The jaws are closed about tissue and ablative energy is applied to each of the ablative elements in the jaws to form a substantially continuous lesion about the clamped tissue. The second jaw is removed or reconfigured away from the first jaw and the first jaw is applied to a second portion of tissue at the surgical site to form a linear lesion thereupon.

In another embodiment, an ablation apparatus comprises a first microwave antenna for forming a first electromagnetic field and a second microwave antenna for forming a second electromagnetic field, with the first and the second antennae supported relative to each other to produce a substantially uniform longitudinal tissue-ablating field in response to tissue-ablating energy applied to the antennae.

These and other advantages and features of the invention will become apparent to those persons skilled in the art upon reading the details of the devices and methods as more fully described below.

### BRIEF DESCRIPTION OF THE DRAWINGS

FIG. 1 is a pictorial illustration of a human heart displaying a bilateral lesion pattern (posterior view);

FIG. 2A is a side view of a surgical clamp for forming an encircling lesion attached to a support structure in accordance with an embodiment of the invention;

FIGS. 2B-2D are side views of surgical clamps for forming linear lesions attached to support structures in accordance with embodiments of the invention;

FIG. 3 is a side view of a surgical clamp including a clamp control element 28 in accordance with an embodiment of the invention;

FIG. 4A is a view of a surgical clamp including a sensor in accordance with an embodiment of the invention;

FIG. 4B is a simplified circuit diagram of a surgical system for performing and detecting ablation in accordance with an embodiment of the invention;

FIGS. 5A and 5B are graphs depicting the radiative field generated by antennae in accordance with an embodiment of the invention;

FIG. 5C is graph depicting the cumulative radiative field generated by the antennae in FIGS. 5A and 5B in accordance with an embodiment of the invention;

FIG. 5D is a side view of a frame for supporting antennae for generating the fields depicted in FIGS. 5A and 5B in accordance with an embodiment of the invention;

FIGS. 6 and 7 are graphs depicting the radiative field generated by antennae in accordance with an embodiment of the invention;

FIG. 8 is graph depicting the cumulative radiative field generated by the antennae in FIGS. 6 and 7 in accordance with an embodiment of the invention;

FIG. 9 is a side display of a frame for supporting antennae for generating the fields depicted in FIGS. 6 and 7 in accordance with an embodiment of the invention;

FIGS. 10-16 are pictorial illustrations of a human heart during various stages of the formation of a “box” lesion around the pulmonary veins of the heart (posterior view); and

FIG. 17 comprises a flow chart illustrating a surgical procedure according to the present invention.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a jaw” includes a plurality of such jaws and reference to “the vein” includes reference to one or more veins and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

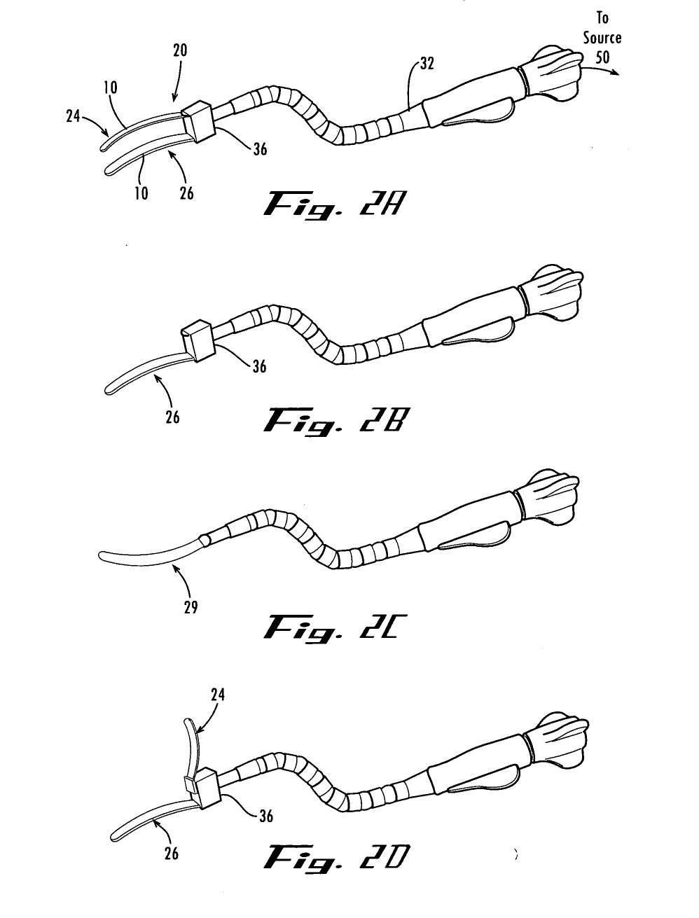

Referring now to FIG. 2A, there is shown a side view of a surgical clamp 20 in accordance with an embodiment of the invention. The clamp 20 comprises a first jaw 24, a second jaw 26 and an attachment portion 36 disposed to attach the jaws 24, 26 of the clamp 20 to the distal end of a support structure 32. Each of the jaws 24, 26 contains an ablation element 10 for ablating cardiac tissue that is positioned adjacent to the jaws.

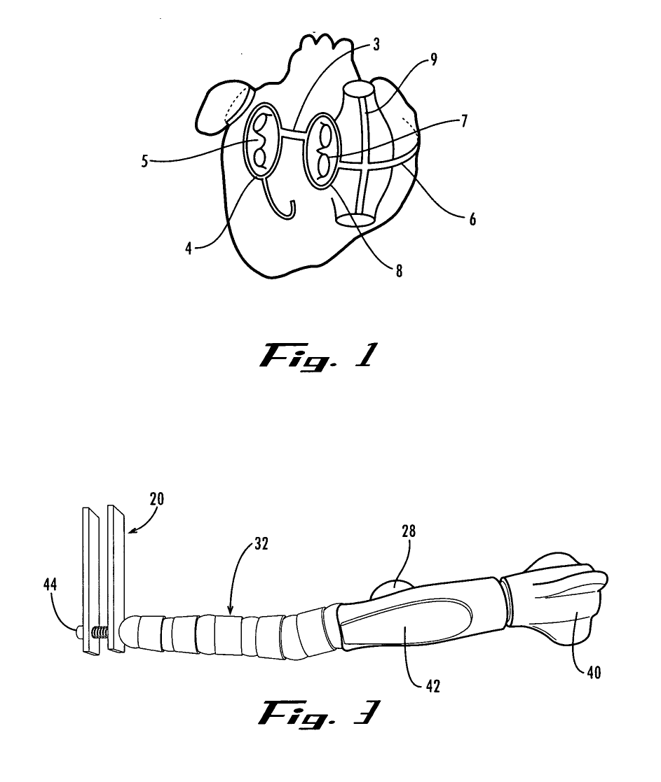

The clamp 20 as shown is capable of being used in a “clamp ablation” mode to make a continuous encircling lesion in response to ablating energy applied to the tissue-ablating elements 10 within the jaws. For example, clamp 20 may be placed around the left pulmonary vein ostia 5 of a human heart and compressed, and the elements 10 within the jaws 24, 26 of the clamp 20 are energized to form an encircling lesion 4 such as shown in FIG. 1. One of the jaws 24, 26 may effectively be removed from the clamp 20, for example as shown in FIG. 2B. The remaining single jaw 26 can be used in a “linear ablation mode” to further ablate tissue in a substantially linear fashion. Operations of various clamp configurations in linear ablation mode are discussed in more detail later herein with reference to FIGS. 2B-2D.

Returning to FIGS. 1 and 2A, the jaws 24, 26 are curvilinear and substantially parallel to each other. In other embodiments, however, the jaws 24, 26 may be shaped differently, for example, to resemble a forcep or surgical grasper. The jaws 24, 26 are substantially rigid and may be formed from biocompatible metals and/or polymers typically used in such an environment, or other biocompatible material. The jaws 24, 26 may be substantially hollow to facilitate installation therein of ablating elements 10. Portions of jaws 24, 26 may be formed of electrically insulating material in order to prevent undesirable electrical conduction to adjacent organs or tissue. Each jaw can accommodate an ablation element coupled to an energy source 50 through, for example, a coaxial cable (not shown) in support structure 32. The energy source 50 may comprise a source of ablating energy, such as, for example, an electrical source for resistance heating, a radiofrequency source, a microwave source, an ultrasonic source, a laser source, or the like. Alternatively, a cryogenic or other source may be used to ablate the tissue, powered by liquid nitrogen or other circulating refrigerant.

In one embodiment, an ablation element 10 comprises a microwave antenna disposed within a hollow chamber or recess within the first jaw 26. The jaw 26 is formed of an appropriate thickness and composition of material to pass the ablating energy for desiccating adjacent tissue. The antenna is positioned within the jaw 26 in order to emit ablative energy along substantially the entire length of the jaw 26. One or more of the jaws 24, 26 may include other surgical elements such as a sensor for measuring a characteristic of tissue in contact therewith.

The clamp 20 of FIG. 2A is attached to the distal end of support structure 32 via the attachment portion 36 of clamp 20. In other embodiments, a connecting rod, shaft, or other structure is used to attach proximal portions of jaws 24, 26 to the distal end of support structure 32. The clamp 20 can be changed from the clamp ablation mode, as shown in FIG. 2A, to a linear ablation mode, as shown in FIGS. 2B-2D. In each of the embodiments illustrated in FIGS. 2B-2D, a single jaw 26 or 29 is shown for performing tissue ablation. The single jaw 26 or 29 may be positioned, for instance, to form a substantially straight ablation line along the circumference of the atria 6, as shown in FIG. 1. Using various mechanisms described below, a single surgical clamp 20 can thus be alternately used to form two different classes of ablation patterns (encircling and linear) on a surgical site.

FIGS. 2B and 2D each show the clamp 20 of FIG. 2A with the second jaw 24 positioned away from the first jaw 26. The removal of the second jaw 24 from proximity to the remaining single jaw 26 precludes contact of the second jaw 24 with tissue and allows the remaining jaw 26 to be applied to a surgical site independently of the second jaw 24 in order to make linear lesions. FIG. 2B shows the second jaw 24 detached entirely from the clamp 20. Any of a variety of detachment mechanisms may be used to convert the clamp 20 from the clamp ablation mode of FIG. 2A to the linear ablation mode of FIG. 2B. For instance, the second jaw 24 may be released, ejected, unscrewed, pulled, or unhooked from the attachment portion 36 of the clamp 20. Alternatively, the second jaw 24 may remain attached to the support structure 32, but be removed from the operational area of the first jaw 26. As shown in FIG. 2D, the second jaw 24 can be rotated away from the first jaw by way of a hinge, gear, ball joint, or like mechanism to facilitate operation of the first jaw 26 in isolation. Although the second jaw 24 as shown in FIG. 2D appears to be rotated substantially in the plane of the two jaws 24, 26 the second jaw 24 may be configured to rotate freely, sidewise, lengthwise, or the like.

In another embodiment, the clamp 20 of FIG. 2A is removed entirely from the support structure 32 and is replaced, as shown in FIG. 2C, with a single jaw 29 to facilitate linear ablation of tissue by the single jaw 29. The two configurations of clamp 20 and single jaw 29 may be used interchangeably by a surgeon over the course of an operation. Using any of the clamps 20 shown in FIGS. 2A-2D, a single structure 32 can thus be used to form various lesion shapes. This simplifies the surgical process while also providing the benefits of a clamp-type ablation device.

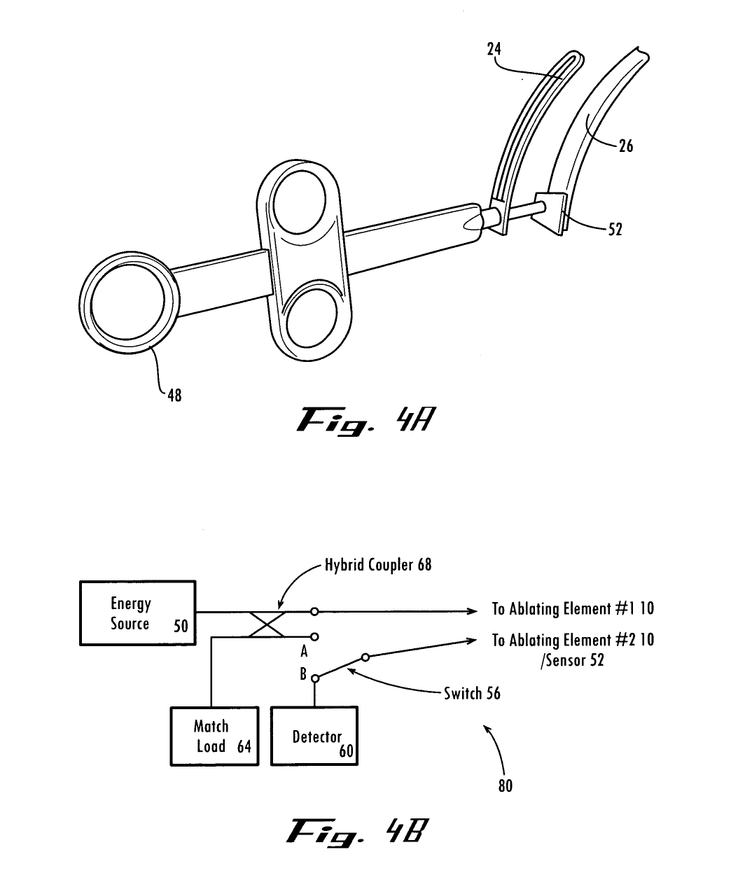

In operation, the surgical clamp 20 of FIG. 2A is attached to the support structure 32 and may be introduced directly onto the patient's heart during open heart surgery. Other clamps 20, such as those shown in FIG. 3 or 4A, may be mounted parallel or perpendicular to, or at an angle to various support structures 32, as desired for specific surgical procedures.

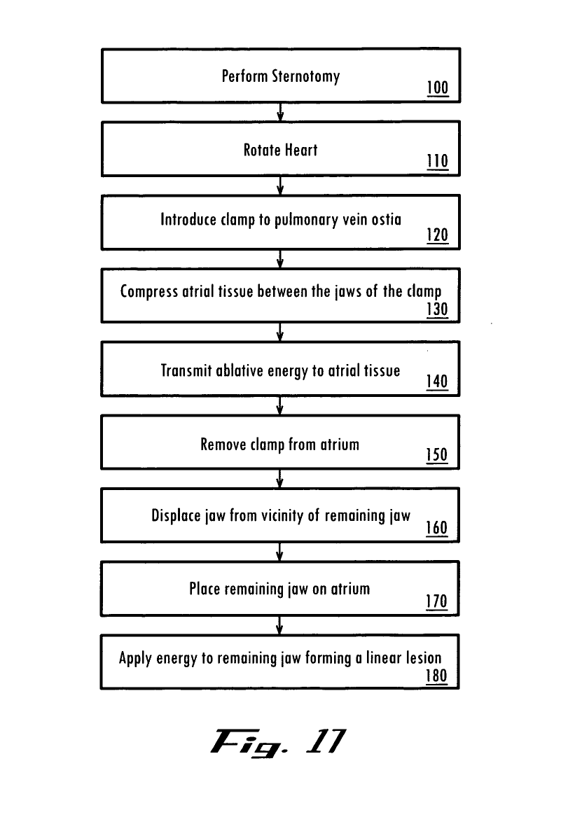

A flowchart of an exemplary surgical procedure performed using surgical clamp 20 is shown in FIG. 17. A partial or full sternotomy (division of the patient's sternum) is performed 100, and the heart is exposed from within the pericardium. The heart is rotated 110 to provide access to the pulmonary veins. Cuts are made as needed and the jaws 24, 26 of the clamp 20 are introduced 120 to the pulmonary vein ostia 5, 7. The jaws 24, 26 are brought together to compress 130 the atrial tissue. Ablative energy is delivered from an energy source 50 by a conductive pathway within the support structure 32 and is transmitted 140 to the tissue via the ablation elements 10 within the jaws 24, 26. After the period of ablation, for example in the case of ablation energy delivered at 65 watts for a period of about 35 seconds, where the tissue to be ablated is about 3 mm to about 5 mm thick (although these specifications may vary under varying conditions such as fat layers present, variations in tissue thickness, variations in tissue conductivity, etc.), the clamp 20 is removed 150 from the atrium, leaving behind a lesion pattern formed by the ablation elements 10. One of the jaws, for instance the second jaw 26, may be displaced 160 from the vicinity of the remaining jaw 24 for instance by rotating the second jaw 26 away from the remaining jaw 24, or removing the jaw 26 entirely from the clamp 20. The remaining jaw 24 can be placed 170 on the atrium by itself, without the second jaw 26. When ablation energy is applied to the remaining jaw 24, a linear lesion is formed 180.

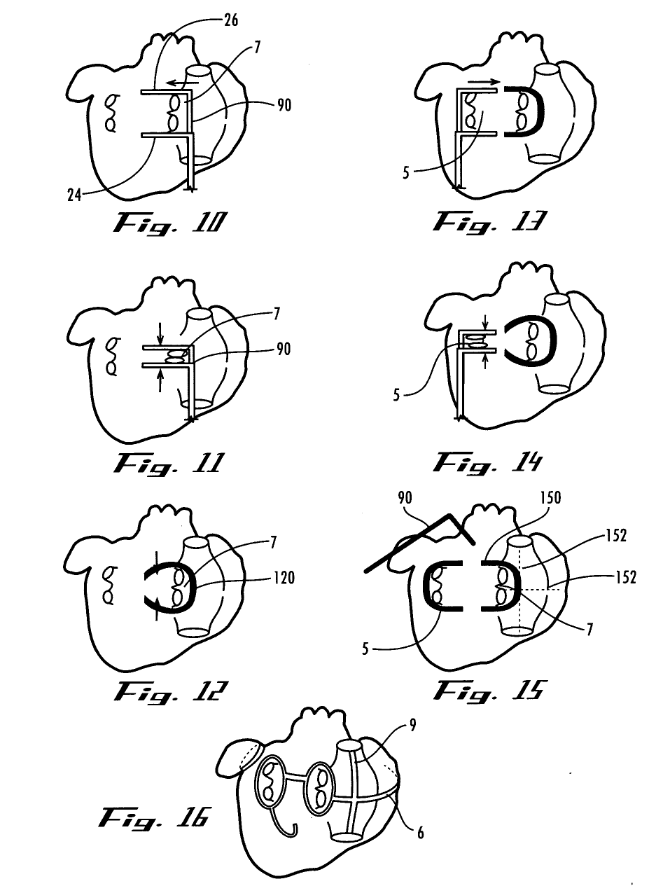

In an open-heart or closed-chest surgical procedure, a clamp 90 can be used to complete a “box” lesion surgical pattern, as shown in the sequence depicted in FIGS. 10-16. After access to the heart has been accomplished, the clamp 90 is placed on the left atrium with the top jaw 26 disposed adjacent to the transverse sinus and the lower jaw 24 adjacent to the oblique sinus, as shown in FIG. 10. The jaws of the clamp 90 are compressed around the ostia of the right pulmonary veins 7, as shown in FIG. 11. After ablation of the ostia 7, the clamp 90 is released and removed, leaving a C-shaped lesion 120 as shown in FIG. 12. The clamp 90 is then placed around the left pulmonary veins 5 as shown in FIG. 13, and compressed, as shown in FIG. 14, to create a second C-shaped lesion. This results in a substantially continuous lesion 150 around the ostia of the four pulmonary veins 5,7, as shown in FIG. 15. To complete the procedure, a jaw of the clamp 90 is removed so that only single jaw 26 remains, and linear lesion patterns are marked 152. The remaining jaw 26 is used to complete the lesion around the vein ostia 5,7 and to form linear lesions around the circumference of the atria 6 and down the length of the aorta 9.

A version of the clamp 20 of FIG. 2A, may be positioned in an ablation cannula for alternative use in various closed-chest surgical procedures. In one embodiment, preparations for cardiac ablation include forming a thoracotomy incision through approximately the third intercostal space in the left anterior chest substantially over the site of the left atrial appendage. Blunt dissection is performed through the intercostal muscle over the pleura, and the cannula is introduced through the left chest toward to the surgical site. Alternatively, a laparoscopic trocar sheath or balloon port may be inserted through the incision to form a port of entry into the left atria while maintaining a sliding seal about the ablation cannula that is inserted into the left atrial appendage.

The jaws 24, 26 of the clamp 20 in ablation clamp mode are positioned about the portions of the heart tissue to be ablated. As described above, the clamp 10 may then be reconfigured to a linear ablation mode to form a required ablation pattern. After tissue ablation is completed about the ostium of each pulmonary vein, the ablation cannula is removed from the atria and the incision therein is sutured closed, or closed with conventional implantable locking clips.

FIG. 3 is a side view of a surgical clamp 20 attached to a support structure 32 including a clamp control element 28 in accordance with another embodiment of the invention. The support structure 32 includes various control structures including a button 42, clamp control element 28, and rotary knob 40 linked to mechanical elements of support structure 32 for controlling the flexible and rigid configuration thereof in a conventional manner. Although the rotary knob 40 is shown mounted to the proximal end of the support structure 32 and the button 42 and clamp control element 28 are shown mounted to proximal portions of support structure 32, one or more of these elements, in combination with other control elements, may be mounted on various portions of the support structure 32. The mechanical parameters controlled by the elements 28, 40, 42 may include the distance between the jaws of the clamp 20, the positioning or detachment of one or more jaws, the flexibility or rigidity of the support structure 32, and the operational mode of the jaws, for example, in sensing or ablating operations modes, as later discussed herein in more detail.

The support structure 32 of FIG. 3 includes interlocking links held together by a tensioning element such as a slidable rod or wire in a conventional manner. The links can be tightened to make the support structure 32 rigid, or loosened to provide maneuverability and flexibility. The tensioning element of the support structure 32 can be controlled by the rotary knob 40. The support structure 32 may also include a retractor system, examples of which are provided in U.S. Pat. Nos. 6,331,158; 6,626,830; 6,885,632 and 6,283,912, each of which is incorporated herein, in its entirety, by reference thereto.

The surgical clamp 20 includes two jaws that are resiliently biased apart in a normally-open position by spring 44. The jaws may be brought together or opened by applying or releasing clamping force on the spring 44 using a manual actuator attached to a clamp control element 28. In another embodiment, the jaws may be brought together by rotation of a knob 40 in a conventional manner or through a pneumatic or hydraulic pump controlled by the button 42. Other aspects of clamp 20 may be controlled by the element 28, knob 40, or button 42. For instance, the button 42 may control ejection or other reconfiguration of one of the jaws of the clamp 20. Alternatively, the knob 40 or element 28 may position or rotate one or more of the jaws of the clamp 20 away from a surgical site. The element 28 may also be used to control the operation of elements mounted in the jaws of the clamp 20, for example, to ablate or sense parameters of lesions. Thus, the element 28 may select and control energizing of one or both of the jaws, or alternating between ablating and sensing modes, or the like.

FIG. 4A is a view of a surgical clamp including a sensor 52 in accordance with an embodiment of the invention. The clamp 20 is attached to a handle 48 of a common configuration in surgical instruments to ease placement of the clamp 20 on a surgical site. One or more sensors 52 can be mounted directly to the inner surface of the jaws 24, 26, as shown. Alternatively, a sensor 52 can be inserted into a grooved portion of one or both of the jaws 24, 26 for removal therefrom at the end of a surgical operation. In one embodiment, the sensor 52 is disposable and comprises a thermochromic liquid crystal (TLC) mounted on a strip-like surface to irreversibly change color in response to attaining a critical temperature (T_{c}), for instance, 50 degrees centigrade, during contact with tissue being ablated. In operation, the strip 52 is placed on one jaw 24 of the clamp to contact one side of tissue being ablated by energy emitted from the other jaw of the clamp 26 disposed on an opposite side of the tissue being ablated. The temperature of the tissue portion is measured by the TLC strip 52 which changes color at T_{c} to confirm necrosis of the tissue being ablated. The TLC strip 52 can be removed from the clamp 20 after surgery, to be kept for future reference or records.

Other sensors may be used to assess tissue ablation, for use with or without a clamp. FIG. 4B is a simplified circuit diagram of a surgical system 80 operable in an ablation mode and a sensing mode in accordance with one embodiment of the invention. A detector 60 is coupled to a sensor 52 by the circuitry shown with switch 56 in the “B” position. The detector processes signals from the sensor 52 and provides a reading based on the signals. The detector 60 can comprise a temperature sensor, calorimeter, power detector, impedence detector, phase detector, or other electrical, optical or like monitoring device, and may be placed in a location remote from the surgical site. The sensor 52 is operable with the detector 60, and can comprise an electrode, optical probe, or other such monitoring implement.

Tissue adjacent to the sensor 52 may be ablated, for example, by an ablating element 10 mounted in one or more jaws of a clamp 20, or by an ablation probe or other energy source. As living tissue is ablated, its physical and electrical properties change in color, temperature, resistance, capacitance, and inductance. A change in color, for instance can be sensed by a colorimeter to indicate that the tissue reached a predetermined temperature characteristic of the color attained. Similarly, a thermal sensor can be used to monitor the temperature of adjacent tissue to enable a surgeon to control application of ablation energy for a set period of time after a critical tissue temperature is reached. The electrical properties of tissue may also be detected by sensor 52. Alternating signal applied to the tissue by an electrode in contact with, or in close proximity to tissue can be used to gauge the completeness of ablation in a known manner. For example, the phase shift of a detected signal relative to an applied alternating current as measured by detector 60 will change over the course of tissue desiccation and will stabilize once necrosis has occurred. By observing such phase-shift characteristics, a surgeon can determine when ablation is complete. As yet another example, the ablation of tissue also causes a loss in water and change in dielectric constant. The rate of change of the dielectric constant usually decreases as the tissue becomes desiccated to provide another measure of transmurality for a surgeon or practitioner to observe.

During surgery, the medical device of the present invention can be used to both perform and monitor ablation. Using the surgical system 80 of FIG. 4B, a clamp including first and second ablating elements 10 can simultaneously energize two portions of heart tissue with the switch in the “A” position as energy is delivered from the power source 50 to both of the ablating elements 10 through a hybrid or directional coupler 68. The ablating elements 10 may be disposed in clamp 20 or other support device. Alternatively, a grounding match load 64 is connected to the power source 50 through the hybrid coupler 64 in place of an ablative element 10 with the switch in the “B” position. In this circuit configuration, the sensor 52 (which may include components of the ablative element 10) senses a characteristic of the tissue ablated by or adjacent to ablating elements 10, as described above. A surgeon can manually transition between the A and B circuit configurations, or, in one embodiment of the present invention, the surgical system 80 can be set up to automatically, intermittently measure the temperature, color, electrical characteristics, or other parameter of the tissue during ablation.

Tissue-ablating energy may include microwave radiation delivered by a microwave antenna that radiates an electromagnetic field about the axis of the antenna. A reflector is positioned to reflect a major portion of the energy from the antenna toward a single direction to make the antenna substantially unidirectional in operation. One difficulty associated with this arrangement is that the intensity or density of emitted energy is non-uniformly distributed along the length of the antenna.

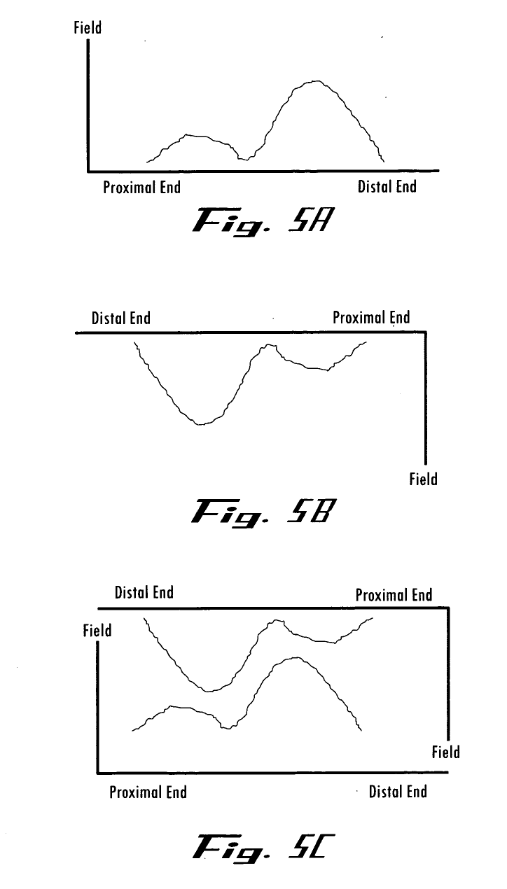

In accordance with one embodiment of the present invention, two antennae that produce substantially complementary distributions of energy density along the length thereof are positioned in adjacent array to produce a cumulative field strength that is more uniformly distributed along the combined lengths of the antennae. For example, the radiation field pattern shown in FIG. 5A generated by a first unidirectional antenna varies in intensity over the length thereof. A lesion formed in tissue at the distal end of such antenna will likely form faster than one created at the proximal end of the antenna. However, flipping an antenna of FIG. 5A end-for-end creates a radiation field, shown in FIG. 5B, substantially complementary to the radiation field of FIG. 5A. Combining the radiation fields of such antennae, as shown in FIG. 5C, creates a more uniform radiation pattern. To form such a combined radiation field, antennae 82, 84 are mounted to or in a clamp or other fixture, as shown in FIG. 5D.

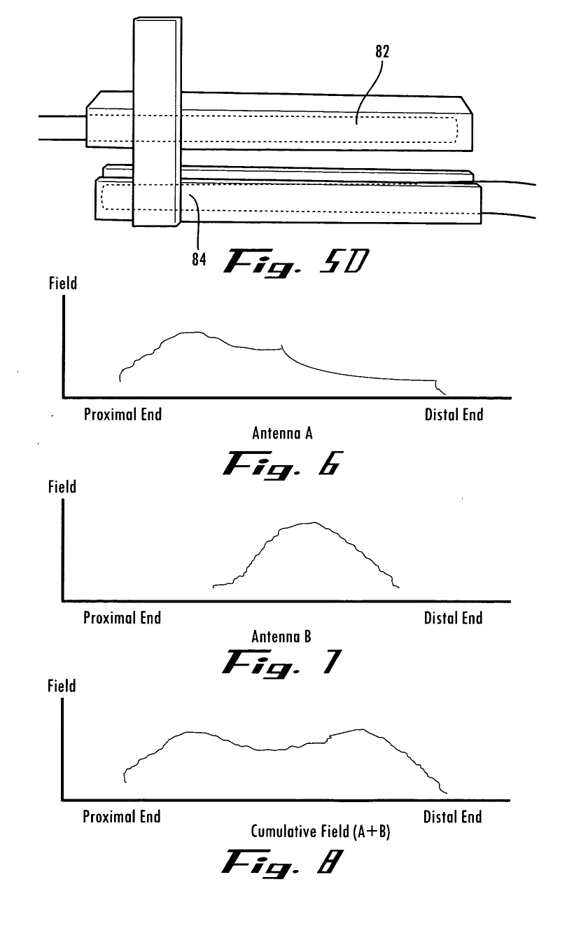

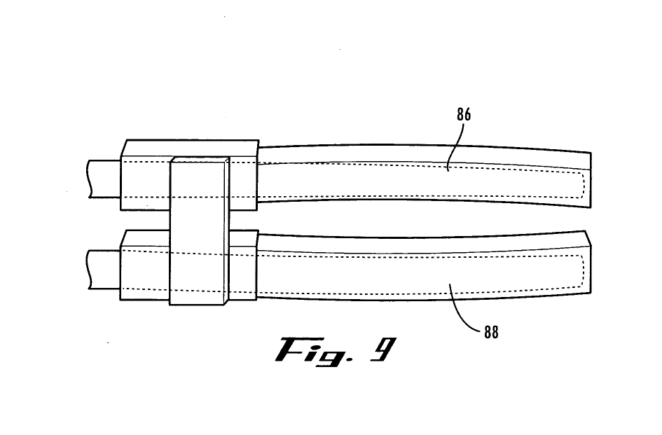

The fields of two such antennae can be combined in other complementary ways to produce a combined field of substantially uniform strength or density along the combined lengths thereof. For instance, the field produced along antenna A as shown in FIG. 6, and the field produced along antenna B as shown in FIG. 7, are both substantially non-uniform but are complementary with respect to each other along the combined lengths thereof. Mounting the antennae 86, 88 in the fixture shown in FIG. 9 produces the cumulative field of more uniform intensity along the combined lengths thereof, as shown in FIG. 8.

Therefore, the tissue-ablation apparatus and procedures according to embodiments of the present invention enable simpler and more efficient ablation of cardiac tissue using apparatus that can be alternately used to make clamp and linear lesions. In addition, assessment apparatus including a thermochromic element such as a liquid crystal material that irreversibly changes color at a critical temperature, may be used to confirm tissue necrosis. And, microwave antennae are positioned to provide a more uniform tissue-ablating energy field along the length of the antennae for forming more uniform tissue lesions.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A surgical clamp having a proximal end for forming a cardiac lesion, the clamp comprising:
a first jaw including a first tissue-ablating element disposed to selectively ablate tissue in proximity thereto; and
a second jaw detachably coupled to the first jaw at a location near a proximal end of the first jaw and adjustable in distance therefrom.

2. The surgical clamp of claim 1 wherein:
the second jaw includes a second tissue-ablating element disposed to selectively ablate tissue in proximity thereto;
the first and second jaws being configurable to compress a tissue structure therebetween in a closed position, and to ablate tissue adjacent to the tissue-ablating elements responsive to the application of ablating energy to selected ones of the first and second tissue-ablating elements; and
the first tissue-ablating element in the first jaw being operable independently of the second tissue-ablating element in the second jaw to form a linear lesion in tissue adjacent thereto responsive to the application of ablating energy to the first tissue-ablating element.

3. The surgical clamp of claim 1 further including:
a spring disposed between the first and second jaws and configured to compress responsive to a decrease in the distance therebetween for resiliently biasing the first and second jaws toward spaced-apart orientations.

4. The surgical clamp of claim 1 further including an attachment portion located near the proximal ends of the jaws for attachment of the clamp to a distal end of a support structure for positioning the clamp with respect to a surgical site.

5. The surgical clamp of claim 1 in which the distance between the first and second jaws is adjustable.

6. The surgical clamp of claim 4 in which the attachment portion is disposed to detach the second jaw from the clamp.

7. The surgical clamp of claim 4 wherein the support structure comprises:
a flexible elongated body extending between a proximal portion and a distal portion that is coupled to the clamp; and
a manual controller mounted to the elongated body for selectively inhibiting flexible movement of the elongated body.

8. The surgical clamp of claim 7 wherein the manual controller comprises a rotatable knob mounted adjacent to the proximal portion of the elongated body and linked to a tensioning member disposed within the elongated body for manually tensioning the tension member to inhibit flexible movement of the elongated body.

9. The surgical clamp of claim 4 wherein the support structure comprises a clamp control element mounted near the proximal end for implementing one of: positioning the clamp in relationship to a surgical site, adjusting a distance between the two jaws of the clamp, and detaching the second jaw from the clamp.

10. The surgical clamp of claim 1 further comprising a sensor disposed in one of the first and second jaws for sensing a characteristic of tissue adjacent thereto.

11. The surgical clamp of claim 10 wherein the sensor is positioned in one of the first and second jaws to sense ablation of tissue in response to tissue-ablating energy applied thereto from the other of the first and second jaws.

12. The surgical clamp of claim 10 wherein the sensor is configured to change color responsive to attainment of an elevated temperature by tissue located adjacent thereto.

13. The surgical clamp of claim 12 wherein the sensor is configured to change color irreversibly responsive to attainment of a selected elevated temperature.

14. The surgical clamp of claim 10 wherein the sensor is responsive to one of the characteristics of: the color of tissue, the impedence of tissue, and the power transmitted through tissue.

15. The surgical clamp of claim 10 wherein:
one of the tissue-ablating elements operates in an ablation mode for delivering ablation energy from an energy source to tissue adjacent to the element, and operates in a sensing mode for monitoring a selected characteristic of ablated tissue adjacent to the one of the tissue-ablating elements.

16. The surgical clamp of claim 15 including circuitry configured to alternate between the ablation mode and the sensing mode for ablating and monitoring tissue.

17. The surgical clamp of claim 1 wherein:
the first tissue-ablating element comprises a first microwave antenna disposed to produce a first electromagnetic field upon energization thereof; and
the second jaw comprises a second microwave antenna positioned to produce a second electromagnetic field substantially complementary to the first electromagnetic field upon energization thereof.

18. A surgical procedure for forming a lesion on a patient's heart using a surgical clamp including two jaws, each jaw including an ablative element disposed along the length of the jaw, the procedure comprising:
forming an incision;
advancing the surgical clamp through the incision toward a surgical site; positioning the surgical clamp adjacent to a portion of the patient's left atrium;
enclosing between the pair of jaws a portion of the ostia of a first pair of the patient's pulmonary veins;
closing the jaws of the clamp and applying ablative energy to each of the ablative elements to form a first substantially continuous lesion;
repositioning the clamp to enclose between the pair of jaws the ostia of a second pair of the patient's pulmonary veins
closing the clamp and applying ablative energy to each of the ablative elements to form a second substantially continuous lesion; and
forming at least one intermediate lesion between the substantially continuous first lesion and the second substantially continuous lesion surrounding a plurality of the patient's pulmonary veins.

19. The surgical procedure of claim 18, wherein the at least one intermediate lesion is formed by the clamp.

20. A surgical procedure for forming a plurality of lesions on a patient's heart using a single surgical clamp including a first and second jaw, each jaw including an ablative element disposed along the length of the jaw, the procedure comprising:
forming an incision;
advancing the surgical clamp through the incision toward a surgical site; positioning the surgical clamp on a first portion of tissue at the surgical site;
enclosing the first portion of tissue between the jaws;
closing the jaws and applying ablative energy to each of the ablative elements to form a substantially continuous lesion in the enclosed portion of tissue;
configuring the second jaw away from the first jaw to facilitate operation of the first jaw independent of the second jaw on a second portion of tissue at the surgical site;
positioning the first jaw on the second portion of tissue; and
forming a linear lesion on the second portion of tissue responsive to the application of ablative energy to the ablative element included in the first jaw.

21. The procedure of claim 20, wherein configuring comprises one of detaching the second jaw and adjusting the second jaw away from the first jaw.

22. The surgical procedure of claim 20, wherein one of the first and second substantially continuous lesions is formed substantially C-shaped.

23. The surgical procedure of claim 20 further comprising monitoring a selected parameter of the ablated tissue.

24. The surgical procedure of claim 23, wherein monitoring includes observing a change in one of: the temperature of tissue, an electrical property of tissue, and the color of tissue.

25. An ablation apparatus comprising:
a first elongated microwave antenna for forming a first electromagnetic field along the length thereof;
a second elongated microwave antenna for forming a second electromagnetic field along the length thereof; and
an element supporting the first and the second antennae relative to each other to produce a substantially uniform combined tissue-ablating field along the lengths thereof responsive to energization thereof.

26. The ablation apparatus of claim 25 wherein:
the first and second antennae are adjacently positioned lengthwise to each other; and
responsive to energization of the antennae, a majority of the tissue-ablating energy is directed between the antennae to form a substantially uniform tissue-ablating field along and between the lengths of the antennae.

27. The ablation apparatus of claim 25, wherein:
the first and the second antennae are disposed to produce substantially similar electromagnetic fields of tissue-ablating energy; and
the antennae are oppositely oriented relative to each other.

28. The ablation apparatus of claim 25, wherein:
the first and the second antennae are spaced apart and are disposed to produce substantially complementary electromagnetic fields of tissue-ablating energy between and along the lengths of the first and second antennae.

29. The ablation apparatus of claim 25, further comprising:
a sensor disposed for sensing a change in a selected characteristic of tissue adjacent to an antenna.

30. The ablation apparatus of claim 29 wherein the sensor senses a characteristic of tissue selected from the group consisting of: the color of tissue, the temperature of tissue, and an electrical parameter of tissue.

31. The ablation apparatus of claim 29, wherein:
a selected one of first and second microwave antenna operates in a first mode for delivering energy through the antenna to tissue adjacent thereto, and operates in a second mode for monitoring the selected characteristic through said one microwave antenna.

32. The ablation apparatus of claim 29, further comprising:
a switch positioned between the selected one of first and second antennae and an energy source for selectively alternating between the first and second mode.

33. The ablation apparatus of claim 31 configured to operate alternately in the first and second modes during the ablation of tissue for assessing ablation thereof.

34. A surgical clamp for forming a cardiac lesion, the clamp comprising:
first and second jaws, each including a tissue-ablating element positionable at a surgical site and disposed to selectively ablate adjacent tissue;
an attachment portion supporting the first and second jaws in clamping configuration and dispose to selectively displace the second jaw from the clamping configuration for isolating the first jaws to ablate tissue adjacent thereto.

35. The surgical clamp as in claim 34, wherein the clamp is operable in a clamp ablation mode with the two jaws disposed to confine for forming a lesion therein in response to applied tissue-ablating energy; and in a linear ablation mode the first jaw isolated from the second jaw and operable to form a linear lesion on tissue adjacent thereto in response to applied tissue-ablating energy.

36. The surgical clamp as in claim 35, in which the isolation is performed selectively by one of: detaching the second jaw from the clamp; and positioning the second jaw substantially away from the first jaw.
